(19) United States
(12) Patent Application Publication
(10) Pub. No.: US 2002/0160385 A1
(43) Pub. Date: Oct. 31, 2002

(54) Methods for associating quantitative traits with alleles in sibling pairs

(76) Inventors: Joel S. Bader, Stamford, CT (US); Aruna Bansal, Cambridgeshire (GB); Pak Sham, London (GB)

Correspondence Address: MINTZ, LEVIN, COHN, FERRIS,, GLOVSKY and POPEO, P.C., One Financial Center, Boston, MA, 02111, US

(21) Appl. No.: 09/999,706

(22) Filed: Oct. 31, 2001

Related U.S. Application Data
(60) Provisional application No. 60/244,444, filed on Oct. 31, 2000.

(51) Int. Cl. C12Q 1/68

(52) U.S. Cl. 435/6

(57) ABSTRACT

Identifying the genetic components of complex diseases is one of the most important goals of the human genome project. These diseases and their underlying risk factors are often better described by quantitative phenotypes than by an arbitrary distinction between affected and unaffected individuals. Association studies are able to identify genetic loci contributing to these quantitative trait loci directly at the cost of requiring large population sizes. Studies of sib pair populations have been suggested to increase power when populations are stratified, and tests on pooled DNA may reduce the experimental burden, but these approaches have been analyzed primarily in the context of affected/unaffected disease phenotypes. Disclosed herein are efficient methods for QTL mapping using DNA pooled from sib pairs. A preferred test using a single set of pools is to select unrelated sibs with extreme phenotypic values, requiring a population size approximately 1.5× larger than for individual genotyping. A preferred strategy overall, with a population size requirement only 1.24× larger than for individual genotyping, is a combined test of DNA pooled according to sib-mean and sib-difference phenotypic values. The optimal pooling fraction is 27% and is insensitive to all model parameters including allele frequency, inheritance mode, and the magnitude of the QTL effect.

### RELATED APPLICATIONS

This application claims priority from U.S. Ser. No. 60/244,444 filed Oct. 31, 2000 which is incorporated herein in its entirety.

### FIELD OF THE INVENTION

The invention relates to a method for detecting an association in a population of individuals between a genetic locus or loci and a quantitative phenotype.

### BACKGROUND OF THE INVENTION

The complex diseases that present the greatest challenge to modem medicine, including cancer, cardiovascular disease, and metabolic disorders, arise through the interplay of numerous genetic and environmental factors. One of the primary goals of the human genome project is to assist in the risk-assessment, prevention, detection, and treatment of these complex disorders by identifying the genetic components. Disentangling the genetic and environmental factors requires carefully designed studies. One approach is to study highly homogenous populations. A recognized drawback of this approach, however, is that disease-associated markers or causative alleles found in an isolated population might not be relevant for a larger population. An attractive alternative is to use well-matched case-control studies of a more diverse population. A second alternative is to study siblings, inherently matched for environmental effects.

Linkage analysis, a traditional method for identifying the genes responsible for a monogenic disorder by identifying genetic markers in linkage disequilibrium with a particular phenotype, loses power for complex disorders because no single disease-related gene is expected to have large penetrance. A more recent approach is to search for alleles whose inheritance is associated or correlated with changes in a phenotypic value. Single nucleotide polymorphisms, or SNPs, can provide such a marker set. These are typically bi-allelic markers with linkage disequilibrium extending an estimated 10,000 to 100,000 nucleotides in heterogeneous human populations. Tens to hundreds of thousands of these closely-spaced markers are required for a complete scan of the 3 billion nucleotides in the human genome. Because each SNP constitutes a separate test, the significance threshold must be adjusted for multiple hypotheses (p-value˜10⁻⁸) to identify statistically meaningful associations. Consequently, hundreds to thousands of individuals are required for association studies.

The population size required for an association test may be reduced by limiting the effect of confounding factors, such as environmental effects or spurious association with markers correlated with ethnicity. Case-control studies, which are used to increase the homogeneity of a test population for studies of diseases with a clear distinction between affected and unaffected, are less applicable to quantitative phenotypes. An alternative has been to conduct genetic studies in highly homogenous populations. A drawback of this approach, however, is that disease-associated markers or causative alleles found in an isolated population might not be relevant for a larger population. A second and more attractive alternative is to use a test population composed of siblings.

The more powerful tests of association require that each individual be genotyped for every marker and remain far too costly for all but testing candidate genes. An alternative that circumvents the need for individual genotypes, related to previous DNA pooling methods for determination of linkage between a molecular marker and a quantitative trait locus, is to determine allele frequencies for sub-populations pooled on the basis of a qualitative phenotype. Populations of unrelated individuals, separated into affected and unaffected pools, have greater power than related populations. If a population consists of sib-pairs, concordant pairs versus unrelated controls have greater power than discordant pairs separated into affected and unaffected pools. Nevertheless, discordant designs might provide a better control for confounding factors such as age, ethnicity, or environmental effects.

The phenotypes relevant for complex disease are often quantitative, however, and converting a quantitative score to a qualitative classification represents a loss of information that can reduce the power of an association study. The location of the dividing line for affected versus unaffected classification, for example, can affect the power to detect association. Furthermore, pooling designs based on a comparison of numerical scores are not even possible with a qualitative classification scheme. These distinctions can be especially relevant when populations contain related individuals and qualitative tests have a disadvantage.

There remains a need for procedures that provide phenotype associations with diseases or pathologies based on phenotypes that may be ranked on a quantititative scale. In such a scheme there is a strong need to identify procedures for optimally obtaining samples, or pooling, from a subpopulation that provide the highest assurance of displaying associations that are present. In addition there is a need to distinguish among various pooling strategies that may arise in cases with different allele frequencies and different allele correlations. There is a further need to devise a test criterion for establishing the significance of associations between phenotypes and diseases or pathologies that may arise. The present invention addresses these and related deficiencies that currently exist.

### SUMMARY OF THE INVENTION

The invention provides a method for detecting an association in a population of individuals between a genetic locus and a quantitative phenotype, wherein two or more alleles occur at the locus, and wherein the phenotype is expressed using a numerical phenotypic value whose range falls within a first numerical limit and a second numerical limit. The method includes: (a) obtaining the phenotypic value for each individual in the population; wherein said population comprises sibling pairs; (b) selecting a first subpopulation of individuals having phenotypic values that are higher than a predetermined lower limit and pooling DNA from the individuals in the first subpopulation to provide an upper pool; (c) selecting a second subpopulation of individuals having phenotypic values that are lower than a predetermined upper limit and pooling DNA from the individuals in the second subpopulation to provide a lower pool; (d) for one or more genetic loci, measuring the difference in frequency of occurrence of a specified allele between the upper pool and the lower pool; and (e) determining that an association exists if the allele frequency difference between the pools is larger than a predetermined value.

In some embodiments, the phenotypic value is obtained as a numerical combination of other phenotypic values. For example, the phenotypic value can be obtained from regressing out the effect of age. In some embodiments, the phenotypes are numerical rankings.

Preferably, the lower limit and the upper limit are chosen such that, for a specified false-positive rate, the frequency of occurrence of false-negative errors is minimized.

In some embodiments, the population comprises unrelated individuals.

Preferably, the predetermined lower limit is set so that the upper pool includes the highest 35% of the population and the predetermined upper limit is set so that the lower pool includes the lowest 35% of the population. For example, in some preferred embodiments the predetermined lower limit is set so that the upper pool includes the highest 30% of the population and the predetermined upper limit is set so that the lower pool includes the lowest 30% of the population. In more preferred embodiments the predetermined lower limit is set so that the upper pool includes the highest 27% of the population and the predetermined upper limit is set so that the lower pool includes the lowest 27% of the population.

If desired, each family is considered as a unit, and either (i) both sibs are selected for the upper pool; (ii) both sibs are selected for the lower pool; or (iii) neither sib is selected. For example, in preferred embodiments selection is based on the mean phenotype of the two sibs. Selection can be based on both sibs being above a threshold or below a threshold.

In some embodiments, the individuals in the population are sibling pairs and each pair is ranked according to a mean value of the phenotypic values of the siblings in each pair, and for sibling pairs that are in a pool, both members of the sibling pair are in the same pool. The predetermined lower limit is set, e.g., so that the upper pool includes the pairs with highest 35% of the mean values in the population and the predetermined upper limit is set so that the lower pool includes the lowest 35% of the mean values in the population. In other embodiments, the predetermined lower limit is set so that the upper pool includes the highest 30% of the mean values in the population and the predetermined upper limit is set so that the lower pool includes the lowest 30% of the mean values in the population, or the predetermined lower limit is set so that the upper pool includes the highest 27% of the mean values in the population and the predetermined upper limit is set so that the lower pool includes the lowest 27% of the mean values in the population.

Preferably, each sib-pair is considered as a unit, and either (i) one sib is selected for the upper pool, and the other sib is selected for the lower pool; or (ii) neither sib is selected. In more preferred embodiments, selection is based on the magnitude difference between sib phenotype values. For example, selection can be based on one sib being above a threshold and the other sib being below a threshold.

In another embodiment, the individuals in the population are sibling pairs, the pairs are ranked by the absolute magnitude of the difference in phenotypic value for the sibs within each pair, the percent of pairs with greatest difference are identified, the percent of pairs being 70%, and the siblings in each pair are distributed such that the sibling with the high phenotypic value is selected for the upper pool and the sibling with the low phenotypic value is selected for the lower pool, providing 35% of the population in each pool.

For example, the percent of pairs can be about 60% and the distribution provides 30% of the population in each pool. In another example, the percent of pairs is 54% and the distribution provides 27% of the population in each pool.

In preferred embodiments, the individuals in the population are sibling pairs The results can be obtained using, e.g., family tests for sib-pairs. Each family is considered as a unit, and either (i) both sibs are selected for the upper pool; (ii) both sibs are selected for the lower pool; or (iii) neither sib is selected. Alternatively, or in addition, each sib-pair is considered as a unit, and either (i) one sib is selected for the upper pool, and the other sib is selected for the lower pool; or (ii) neither sib is selected.

In some embodiments, an unrelated population is selected from a sib-pair population and pooling is conducted on the derived unrelated population. In some preferred embodiments, the sibling with phenotype furthest from the overall mean is selected from each family to generate an unrelated population.

In some embodiments, unrelated individuals are provided by a process comprising the steps of: (a) providing a superpopulation of individuals, each individual being a member of a sibling pair; (b) selecting that member of each sibling pair having a phenotypic value such that the absolute value of the difference between the individual's phenotypic value and either the first numerical limit or the second numerical limit is lower than the difference for the other individual in the pair, thus providing a population of unrelated individuals; (c) setting the predetermined lower limit so that the upper pool includes the highest 36% of the population and the setting the predetermined upper limit so that the lower pool includes the lowest 36% of the population.

In some preferred embodiments, one member of each sibling pair is chosen at random to provide a group of unrelated individuals; and the members of the group having phenotypic values greater than a predetermined lower limit are placed in the first subpopulation and the members of the group having phenotypic values lower than a predetermined upper limit are placed in the second subpopulation.

In some preferred embodiments, only one member of a sibling pair is placed in a subpopulation; wherein the fraction of individuals in the first subpopulation is determined using Equation A and the fraction of individuals in the second subpopulation is determined using Equation B, and wherein the sibling with genotype G₁ is selected for the upper pool if the value of φ is in the interval 0<φ<π/2 or is selected for the lower pool if the value of φ is in the interval π<φ<3π/2 and the sibling with genotype G₂ is selected otherwise.

In some preferred embodiments, the mean phenotypic value for the pair is calculated; and the first subpopulation contains those pairs whose mean phenotypic value is greater than a predetermined minimum value and the second subpopulation contains those pairs whose mean phenotypic value is lower than a predetermined maximum value.

In some preferred embodiments, (i) the difference between the phenotypic values for the members of each sibling pair is calculated; (ii) those sibling pairs whose values of the calculated difference are greater than a predetermined minimum value for the difference are identified; and (iii) in each identified sibling pair, placing the sibling with the higher phenotypic value in the first subpopulation and the sibling with the lower phenotypic value in the second subpopulation.

In some embodiments, (i) the mean phenotypic value for the pair is calculated; and (ii) a first upper subpopulation contains those pairs whose mean phenotypic value is greater than a predetermined minimum value and a first lower subpopulation contains those pairs whose mean phenotypic value is lower than a predetermined maximum value; (iii) the difference between the phenotypic values for the members of each sibling pair is calculated; (iv) those sibling pairs whose values of the calculated difference of step (iii) are greater than a predetermined minimum value for the difference are identified; and (v) in each sibling pair identified in step (iv), placing the sibling with the higher phenotypic value in a second upper subpopulation and the sibling with the lower phenotypic value in a second lower subpopulation.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In the case of conflict, the present Specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1. The population size required to detect association with a test of pooled DNA is shown as a function of the fraction of population ρ selected for each pool, relative to the population size required for a regression test using individual genotyping, for a QTL making a small contribution to a complex trait. The same family structure and the same phenotypic variable, either the individual phenotype, the sib-mean, the sib-difference, or the combined results from sib-mean and sib-difference tests, are used for tests based on pooling and individual genotyping. All of these tests show the same relative efficiency as a function of pooling fraction, with an optimal fraction of 0.27 requiring only 1.24× the population for individual genotyping. The sib-radial design (referred to as Mahanlobis) is compared to the combined regression test for a sibling phenotypic correlation of t_{R}=0.5. The optimum occurs for this, and all other values of t_{R}, at ρ=0.188.

FIG. 2. The population size required to detect association for the sib-radial design, relative to the population required for a combined regression test using individual genotypes, is shown as a function of the sibling phenotypic correlation t_{R}.

FIG. 3. The number of individuals required for pooling designs with a sib-pair family structure is compared to the number of unrelated individuals for an association test of equivalent power and significance as a function of the sibling phenotypic correlation tR.

FIG. 4. (A) Exact numerical results for the population size required to detect association are shown for pooling designs as a function of σ_{A}²/σ_{R}², the ratio of the additive variance of the QTL to the residual variance. Linearity on this log-log plot indicates that the analytic theory provides an accurate estimate of population size requirements. Deviations are seen only when the variance ratio is 0.1 or higher, more characteristic of a monogenic trait than a complex trait. The sib-radial design (referred to as Mahanlobis) is less robust than the other designs. The remaining parameters are allele frequency 0.1, additive inheritance, type I error 5×10⁻⁸, and type II error 0.2. (B) The allele frequency difference at significance is shown for the same parameters as in FIG. 4A. Experimental measurements of allele frequency differences must provide this level of precision to prevent loss of power. In this figure, unrelated-population is a dotted line, sib-radial (referred to as Mahanlobis) is a thin line, sib-mean is a dashed line, sib-difference is a dot-dashed line, and sib-combined is a thick line.

FIG. 5. Exact numerical results for the population size required to detect association is shown as a function of the allele frequency p for (A) dominant inheritance, (B) additive inheritance, and (C) recessive inheritance for tests using pooled DNA. The variance ratio σ_{A}²/σ_{R}² is 0.02, the type I error is 5×10⁻⁸, the type II error is 0.2, the pooling fraction 0.27 is used for all designs except sib-radial, for which 0.188 is used. The population required to detect association with specified power and significance is almost flat as p decreases below 0.5, then rises sharply asp falls below a critical value dependent on inheritance mode, approximately σ_{A}²/8 for a dominant inheritance, σ_{A}²/2 for additive inheritance, and σ_{A}^{⅔}/2 for recessive inheritance. The sib-radial design is less robust to small allele frequencies than the other designs. In this figure, unrelated-population is a dotted line, sib-radial (referred to as Mahanlobis) is a thin line, sib-mean is a dashed line, sib-difference is a dot-dashed line, and sib-combined is a thick line.

FIG. 6. Exact numerical results for the population size required to detect association is shown as a function of the ratio d/a, describing the inheritance mode, for allele frequencies of (A)p=0.5, (B)p=0.25, and (C)p=0.1 for tests using pooled DNA. Dominant inheritance corresponds to d/a=1, additive inheritance is d/a=0, and recessive inheritance is d/a=−1. All other parameters are as in FIG. 5. In (A), all designs except sib-radial are insensitive to the inheritance mode. In (B), the additive variance vanishes at d/a=−2, corresponding to an over-dominant major allele. The required population size grows much larger for larger values of d/a and is smaller for more negative values. In (C), the additive variance vanishes at d/a=−1.125, with a narrower region where required population sizes are not predicted accurately by analytic theory. In this figure, unrelated-population is a dotted line, sib-radial (referred to as Mahanlobis) is a thin line, sib-mean is a dashed line, sib-difference is a dot-dashed line, and sib-combined is a thick line.

### DETAILED DESCRIPTION OF THE INVENTION

1. Definitions

Glossary of mathematical symbols

X quantitative phenotypic value of an individual

Xᵢ quantitative phenotypic value of sib i, with i=1 or 2 for sib-pairs

X_{±} (X₁±X₂)/2

r phenotypic correlation between sibs

Aᵢ allele inherited at a particular locus. For a bi-allelic marker, i=1 or 2

G genotype at the locus, either A₁A₁, A₁A₂, or A₂A₂ for a bi-allelic marker

Gᵢ genotype for sib i, with i=1 or 2 for sib-pairs

P(G) genotype probability

P(G₁,G₂) joint sib-pair genotype probability

f(X₁,X₂) joint sib-pair phenotype probability distribution

f[X₁,X₂|G₁,G₂] joint sib-pair phenotype probability distribution conditioned on genotypes

p frequency of allele A₁ in a population

q frequency of the remaining alleles, with q=1−p

pᵢ frequency of allele A₁ in sib i, either 1, 0.5, or 0 for an autosomal marker

p_{±} (p₁±p₂)/2

a half the difference in the shift in the mean phenotypic value of individuals with genotype A₁A₁ compared to A₂A₂

d difference in the mean phenotypic value between individuals with genotype A₁A₂ compared to the mid-point of the means for A₁A₁ and A₂A₂

μ mean phenotypic shift due to the locus, equal to a(p−q)+2pqd

σ_{A}² additive variance of phenotype X due to the genotype G

σ_{D}² dominance variance due to the genotype G

σ_{R}² residual phenotypic variance, with σ_{A}²+σ_{D}²+σ_{R}²=1

N the total number of individuals whose DNA is available for pooling

n number of individuals selected for a single pool

ρ pooling fraction defined as n/N

p_{U},p_{L} frequency of allele A₁ in the upper (U) or lower (L) pool

T test statistic, which is expected to be close to zero when the genotype G does not affect the phenotypic value and is expected to be non-zero when individuals with genotypes A₁A₁, A₁A₂, and A₂A₂ have different mean phenotypic values. As formulated here, Thas a normal distribution with unit variance. Under the null hypothesis that σ_{A}=(2pq)^{½}[a−(p−q)d] is zero, the mean of T is zero. Under the alternative hypothesis that σ_{A} is non-zero, the mean of T is also non-zero.

σ₀² variance of n^{½}(p_{U}−p_{L}) under the null hypothesis

σ₁² variance of n^{½}(p_{U}−p_{L}) under the alternative hypothesis

Φ(z) cumulative standard normal probability, the area under a standard normal distribution up to normal deviate z

z_{α} normal deviate corresponding to an upper tail area of α, defined as Φ(z_{α})=1−α

α type I error rate (false-positive rate). For a one-sided test, T>z_{α} corresponds to statistical significance at level α, typically termed a p-value. A typical threshold for significance is a p-value smaller than 0.05 or 0.01. If M independent tests are conducted, a conservative correction that yields a final p-value of α is to use a p-value of α/M for each of the M tests.

β type II error rate (false-negative rate). The power of a test is 1−β.

H(x) Heaviside step function

As used herein, when two individuals are “related to each other”, they are genetically related in a direct parent-child relationship or a sibling relationship. In a sibling relationship, the two individuals of the sibling pair have the same biological father and the same biological mother.

As used herein, the term “sib” is used to designate the word “sibling”, and the sibling relationship is defined above. The term “sib pair” is used to designate a set of two siblings.

The members of a sib pair may be dizygotic, indicating that they originate from different fertilized ova. A sib pair includes dizygotic twins.

The term “quantitative trait locus”, or “QTL”, is used interchangeably with the term “gene” or related terms, including alleles that may occur at a particular genetic locus.

A focus of the present invention is to examine the statistical power of pooling designs for quantitative phenotypes. Five basic types of pooling designs are considered: selecting unrelated individuals, selecting sib pairs for the same pool, selecting sib pairs and splitting each pair between pools in two different ways, and a combined test of the sib-together and sib-apart tests. The selection rules are optimized to minimize the population requirements for each type of design, and the powers of the designs are compared with each other and with individual genotyping.

A variance components model is used as the basis for the optimization of pooling designs. This model describes the joint phenotype-genotype distribution of an unselected population and includes a term specifically describing the phenotypic correlation between siblings. The test statistic used to detect association is the allele frequency difference between two pools. Analytical formulas for population size requirements are derived for a QTL making a small contribution to the phenotypic values, exactly the limit that attains for a complex trait. The validity of the formulas is ascertained by exact numerical computation over a wide range of parameters of the population model, including the effect of the QTL, the frequency of the minor allele and its dominant, recessive, or additive inheritance mode. The sensitivity of the results to the genetic model and the pooling design are also explored with exact numerical computation.

2. Methods

2.1 Variance Components Model

A standard variance components model is used to describe the joint phenotype-genotype probability distribution (Falconer and MacKay 1996). A quantitative phenotype X, standardized to mean 0 and variance 1, is hypothesized to be affected by the genotype G at a biallelic locus with minor allele A₁ and major allele A₂ occurring at population frequencies p and 1−p. More generally, A₂ may represent any of a number of alternate alleles, and 1−p their aggregate frequency. The population is assumed to be random mating and in Hardy-Weinberg equilibrium. The symbol P is used to denote a probability, and the genotype frequencies P(G) are p², 2p(1−p), and (1−p)² for A₁A₁, A₁A₂, and A₂A₂ respectively. The frequency of allele A₁ in genotype G, denoted P_{G}, is 1 for A₁A₁, 0.5 for A₁A₂, and 0 for A₂A₂. The variance of the allele frequency per individual is p(1−p)/2 and is denoted σₚ².

The genotype combination for a sib pair is denoted P(G₁,G₂). The probability distribution P(G₁,G₂) of the 9 possible combinations of dizygotic sib-pair genotypes, shown in Table I, can be derived by considering all possible parental mating types and their offspring genotype distributions (Neale and Cardon 1992). The genetic correlation between sibs implies that P(G₁,G₂)≠P(G₁)P(G₂).

The effect μ(G) of genotype G on the phenotype is a−μ, d−μ, and −a−μ for genotypes A₁A₁, A₁A₂, and A₂A₂ respectively. The constant μ=a(2p−1)+2dp(1−p) ensures that the phenotype has mean 0. The ratio d/a determines the inheritance mode of allele A₁. The ratio is −1 for a recessive allele, +1 for a dominant allele, and 0 for an additive allele. Co-dominance implies a ratio between −1 and +1, while over-dominance implies a ratio with a magnitude greater than 1.

The phenotypic variance contributed by the genotype G can be partitioned into an additive component σ_{A}² and an dominance component σ_{D}², with

σ_{A}²+σ_{D}²=2p(1−p)[a−d(2p−1)]²+4p²(1−p)²d².

Remaining genetic and environmental factors contribute a residual variance σ_{R}²=1−(σ_{A}²+σ_{D}²) to the total phenotypic variance. For a complex trait, σ_{A}²+σ_{D}² are small for any particular QTL, and σ_{R}² is close to 1. The additive variance is typically larger than the dominance variance, even for alleles with a dominance or recessive inheritance mode.

The probability density of phenotypic values for sib pairs is ƒ(X₁,X₂), where the symbol ƒ is used generally to denote a probability density. The unconditional probability density is a mixture of conditional probability densities dependent on the 9 possible sib-pair genotypes,

$$f ( {X}_{1} , {X}_{2} ) = {\sum }_{{G}_{1} {G}_{2}} f ( {X}_{1} , {X}_{2} \vert {G}_{1} , {G}_{2} ) P ( {G}_{1} , {G}_{2} ) .$$

The mean of Xᵢ is μ(Gᵢ) for sib i=1 or 2; both X₁ and X₂ have residual variance σ_{R}²; and the phenotypic correlation between X₁ and X₂ due to shared residual genetic and environmental factors is t_{R}. The total phenotypic correlation t measured for sib pairs is

t=t_{R}+σ_{A}²/2+σ_{D}²/4

when effects from genotype G are included. For a complex trait, t and t_{R} are nearly identical. The distributions ƒ(X₁,X₂) and ƒ(X₁,X₂|G₁G₂) are normalized to 1 for integration over X₁ and X₂.

Although X₁ and X₂ are a natural choice of coordinates for expressing sibling phenotypic values, it is more convenient to introduce other coordinate systems in which the variables are uncorrelated and the probability distributions are separable when the QTL has a small effect.

One choice of separable coordinates is the sib-mean/sib-difference coordinate system, with the sib mean X₊ and the sib difference X₋ expressed in terms of X₁ and X₂ as

X_{±}=(X₁±X₂)/2.

The probability distribution in these coordinates, ƒ(X₊,X_{-|G}₁G₂), separates into the product of ƒ(X₊|G₁,G₂) and ƒ(X₋|G₁,G₂), with

ƒ(X_{±}|G₁,G₂)=(2πσ_{±}²)^{-½}exp{−[X_{±}−μ_{±}(G₁,G₂)]²/2σ_{±}²}, with

μ_{±}(G₁,G₂)=[μ(G₁)±μ(G₂)]/2 and

σ_{±}²32 σ_{R}²(1±t_{R})/2.

The distributions for X₊ and X₋ are normalized to 1. It is also convenient to define sib-mean and sib-difference allele frequencies p_{±}(G₁,G₂) as

p_{±}(G₁,G₂)=(p_{G}_{₁}±p_{G}_{₂})/2.

The variance of the sib-mean and sib-difference variables may be expressed more generally as

Var(X_{±})=(T_{±})σ_{R}² and

Var(p_{±})=(R_{±})σₚ²

where

T_{±}=[1±(s−1)t_{R}]/s,

R_{±}=[1±(s−1)r]/s,

the family size s is 2 for sib-pairs, and the genotypic correlation r is ½ for full sibs.

Radial coordinates are a second choice yielding a separable probability distribution in the absence of the QTL. The radial distance b and phase φ are defined so that

X₊=bσ₋sin φ and

X₋=bσ₊cos φ.

The probability distribution in radial coordinates is

ƒ(b,φ|G₁,G₂)=(2π)⁻¹exp[−(b sin φ−₊)²/2]exp[−(b cos φ−ν₋)²/2] with

ν_{±}=μ_{±}/σ_{±}.

This distribution is normalized as

$${\int }_{0}^{2 π} ⅆ ϕ {\int }_{0}^{∞} ⅆ b b f ( b , ϕ \vert {G}_{1} , {G}_{2} ) = 1.$$

In the absence of a contribution from the QTL, ƒ(b,φ|G₁,G₂) reduces to (2π)⁻¹ exp(−b²/2) and the probability density in polar coordinates is independent of the phase φ. Contour lines of equal probability density in the X₁−X₂ plane are ellipses tilted at 45° with a ratio of major axis to minor axis of [(1+t)/(1−t)]^{½}.

2.2 Pooling Designs

The tests of association described here depend on detecting differences in allele frequency in DNA pooled from individuals chosen from a larger, unselected population of size N, either all unrelated or comprising N/2 sib pairs. We consider balanced designs of two pools with n individuals selected for each pool, and the pooling fraction ρ is defined as n/N.

If the N individuals are unrelated, a particularly simply design is to select the n individuals whose phenotypic values are at the upper and lower tails of the distribution, which defines upper and lower thresholds X_{U} and X_{L}. This design has been analyzed previously in the context of both selection (Ollivier 1997) and association (Bader et al. 2000) studies and is here termed the unrelated-population design.

A corresponding design for sib pairs is termed unrelated-random. In this design, one sib is chosen at random from each sib-ship to generate a population of N/2 unrelated individuals. Individuals at the upper and lower tails of this unrelated population are then selected for pooling. The unrelated-random design for N individuals with pooling fraction p is exactly equivalent to the unrelated-population design for N/2 individuals with pooling fraction 2ρ. The unrelated-random design is not expected to be as powerful as sib-pair methods making use of family structure information.

A second design selecting only unrelated individuals is termed sib-radial. The sib-mean X₊ and sib-difference X₋ are converted to a radial coordinate b according to

b²=2X₊²/(1+t)+2X₋²/(1−t).

The n sib-ships with the largest magnitude b and a positive sib-mean X₊ are identified, and the sibling with the larger phenotypic value is selected for the upper pool. Similarly, the n sib-ships with the largest b and negative sib-mean are identified, and the sibling with the more negative phenotypic value is selected for the lower pool.

Two remaining designs select both individuals within a sib pair. The sib-mean design selects each sib-ship as a family unit based on the phenotypic mean of the pair. The n/2 pairs at the extreme upper and lower tails of the distribution of phenotypic means for sib-ships, comprising n individuals each, are selected for the upper and lower pools. The upper and lower thresholds are again termed X_{U} and X_{L}.

The sib-difference design selects individuals based on the difference of phenotypic values within each sib-ship, or equivalently the within-family phenotypic variance. The n sib-pairs with the greatest variance are identified. Within each family, the individual with the higher phenotypic value is selected for the upper pool, and the individual with the lower phenotypic value is selected for the lower pool. The threshold for the magnitude of the difference |X₁−X₂|/2 is termed X_{T}.

The sib-mean and sib-difference methods are similar to between-family and within-family selection methods for breeding value (Falconer and MacKay 1996), with two notable differences. First, within-family selection is typically applied to large family sizes without reducing the number of families, while here the number of families is reduced according to within-family variance. Second, individuals are selected here both for extreme high and extreme low phenotypic values rather than for values extreme in only one direction. Breeding methods concerned with only a single direction are closer analogs to concordant and discordant selection based on affected/unaffected status (Risch and Teng 1998).

The results of the sib-mean and sib-pair design may be combined as a single, more powerful test, as is commonly done with regression tests for individual genotyping (Abecasis 2000).

2.3 Test Statistic, Significance and Power for Pooling Designs

The test significance for an association study based on pooled DNA is the difference Δp in allele frequency between the frequencies p_{U} and p_{L} measured for each pool. When the number of alleles expected in each pool is large, Δp has a normal distribution with variance defined as σ₀²/n under the null hypothesis and σ₁²/n under the alternative hypothesis. For a one-sided test with type I error rate α, the standard normal deviate z_{α} is defined as 1−α=Φ(z_{α}), where Φ(z) is the standard normal probability distribution for normal deviate z. Values of Δp more extreme than z_{α}σ₀/n^{½} are significant at level α. The type II error rate is β, corresponding to the power 1−β to reject the null hypothesis, and the normal deviate z_{1-β} is defined as Φ⁻¹(β). The population size required to attain power β is

N=(z_{α}σ₀−z_{1-β}σ₁)²/ρE(Δp)²,

where E(Δp) is the expected value of Δp under the alternative hypothesis and the relationship n=ρN has been used.

For a normally distributed test statistic q with expectation 0 under the null hypothesis, expectation E(q) under the alternative hypothesis, and variance Var(q) under both hypotheses, the relationship providing the population size required may also be written

[Var(q)]⁻¹=(z_{α}−z_{1-β})²/[E(q)]².

Unrelated-population Design

For the unrelated-population design, upper and lower thresholds X_{U} and X_{L} are defined so

ρ=Σ_{G}Φ{−[X_{U}−μ(G)]/σ_{R}}P(G) and

ρ=Σ_{G}Φ{[X_{L}−μ(G)]/σ_{R}}P(G).

The population fractions θ_{U}(G) and θ_{L}(G) are defined as

θ_{U}(G)=ρ⁻¹Φ{−[X_{U}−μ(G)]/σ_{R}}P(G) and

θ_{L}(G)=ρ⁻¹Φ{[X_{L}−μ(G)]/σ_{R}}P(G).

The expected allele frequencies under the alternative hypothesis are

E(p_{U})=Σ_{G}θ_{U}(G)p_{G} and

E(p_{L})=Σ_{G}θ_{L}(G)p_{G} with

E(Δp)=E(p_{U})−E(p_{L}).

The expected variance of the test statistic is obtained from the properties of a multinomial distribution (Beyer 1984) as

σ₀²=2{Σ_{G}P(G)p_{G}²}−2p²=2σₚ² and

σ₁²=Σ_{G}[θ_{U}(G)+θ_{L}(G)]p_{G}²−(p_{U}²+p_{L}²).

These equations may be solved numerically for X_{U} and X_{L} as a function of ρ to yield the required population size N. These numerical results, based on a normal distribution for Δp, differ from the exact results for the true multinomial distribution of Δp by no more than 5% when the number of alleles summed over both pools is greater than 60, and by approximately 10% when the number of alleles is 10. For purposes here, the numerical results for normal Δp are considered essentially exact.

An approximate analytical expression for N may be obtained when σ_{R}² is close to 1 by noting that

Φ(z−δ)=Φ(z)−y_{ρ}δ,

correct to lowest order in the small parameter δ, where y_{ρ}=(2π)^{-½}exp{−[(Φ⁻¹(ρ)]²/2} is the standard normal density when the cumulative probability is ρ. Taking μ(G)/σ_{R} as the small parameter δ, the threshold phenotypic values are

X_{U}=−X_{L}=−σ_{R}Φ⁻¹(ρ),

the expected allele frequency difference is

E(Δp)=2y_{ρ}[Σ_{G}P(G)μ(G)p_{G}]/ρσ_{R}=2y_{ρ}σₚσ_{A}/ρσ_{R},

and both σ₀² and σ₁² are 2σₚ². The resulting approximation for the required population size is

Nᵤₙᵣₑₗ₋ₚₒₚₗₙ=(ρ/2y_{ρ}²)(z_{α}−z_{1-β})²σ_{R}²/σ_{A}²,

This functions is a minimum at ρ=0.27, with ρ/2y_{ρ}²=1.24. The trivial extension to the unrelated-random design for sib-pairs yields

Nᵤₙᵣₑₗ₋ᵣₐₙ=2[(2ρ)/2y_{2ρ}²](z_{α}−z_{1-β})²σ_{R}²/σ_{A}²,

twice as large as Nᵤₙᵣₑₗ₋ₚₒₚₗₙ with a pooling fraction half as large.

Sib-radial Design

For the sib-radial design, thresholds b_{U} and b_{L} are established for the upper and lower pool by the normalization equations

$$\begin{matrix}ρ = ( 1 / 2 ) {\sum }_{{G}_{1} , {G}_{2}} P ( {G}_{1} , {G}_{2} ) {\int }_{0}^{π} ⅆ ϕ {\int }_{{b}_{U}}^{∞} ⅆ bbf ( b , ϕ \vert {G}_{1} , {G}_{2} ) \\ and & ( Equation A ) \\ ρ = ( 1 / 2 ) {\sum }_{{G}_{1} , {G}_{2}} P ( {G}_{1} , {G}_{2} ) {\int }_{π}^{2 π} ⅆ ϕ {\int }_{{b}_{L}}^{∞} ⅆ bbf ( b , ϕ \vert {G}_{1} , {G}_{2} ) . & ( Equation B )\end{matrix}$$

The factor of (½) arises because only one sib is selected from each sib-ship, and we have used the approximation that the QTL makes a small contribution to the phenotype correlation t. The sibling with genotype G₁ is selected for the upper pool in the interval 0<φ<π/2 and for the lower pool in the interval π<φ<3π/2; the sibling with genotype G₂ is selected otherwise. The genotype probabilities θ_{U}(G) and θ_{L}(G) for the upper and lower pools may be written

$${θ}_{U} ( G ) = {ρ}^{- 1} {\sum }_{{G}^{′}} P ( G , {G}^{′} ) {\int }_{0}^{π / 2} ⅆ ϕ {\int }_{{b}_{U}}^{∞} ⅆ bbf ( b , ϕ \vert G , {G}^{′} ) and \\ {θ}_{L} ( G ) = {ρ}^{- 1} {\sum }_{{G}^{′}} P ( G , {G}^{′} ) {\int }_{π}^{3 π / 2} ⅆ ϕ {\int }_{{b}_{L}}^{∞} ⅆ bbf ( b , ϕ \vert G , {G}^{′} ) ,$$

where the symmetry between siblings has allowed the change in integration limits for φ to consider only the regions where sibling 1 is selected. Numerical results for the required population size may then be obtained as outlined above for the unrelated-population design.

An analytic approximation for the population size requirement may be obtained by noting that

ƒ(b,φ|G₁,G₂)=(2π)⁻¹(1+bν₊cos φ+bν₋sin φ)exp(−b²/2)

to lowest order in the gene effect μ(G). The normalization condition leads to the equation

ρ=(¼)exp(−b_{ρ}²/2),

with b_{U}=b_{L}=b_{ρ} defined in terms of the pooling fraction ρ. The genotype frequencies in the upper and lower pools are

θ_{U,L}(G)=P(G)±Σ_{G′}P(G,G′)(ν₊+ν₋)[(2b_{ρ}/π)+Φ(−b_{ρ})/ρ(2π)^{½}],

where the upper pool has the + sign and the lower pool the − sign. The expected allele frequencies in the upper and lower pools are

E(p_{U,L})=p±[(2b_{ρ}/π)+Φ(−b_{ρ})/ρ(2π)^{½}][R₊/T₊^{½}+R₋/T₋^{½}]σₚσ_{A}/σ_{R},

where the upper pool has the positive deviation from p and the lower pool the negative deviation. These results are derived using the identities

$${\sum }_{{G}_{1} , {G}_{2}} P ( {G}_{1} , {G}_{2} ) μ ( {G}_{1} ) {p}_{{G}_{1}} = ( 1 / r ) {\sum }_{{G}_{1} , {G}_{2}} P ( {G}_{1} , {G}_{2} ) μ ( {G}_{1} ) {p}_{{G}_{2}} = {σ}_{A} {σ}_{p}$$

where r is the genotypic correlation. Since θ_{U}(G)+θ_{L}(G) is 2P(G), the variance term σ₁² is equal to σ₀², and both are equal to 2σₚ² because all the pooled individuals are unrelated. The approximate expression for the number of individuals required for the sib-radial design is

N_{sib-radial}=(2ρ)⁻¹[(2b_{ρ}/π)+Φ(−b_{ρ})/ρ(2π)^{½}]⁻²[R₊/T₊^{½}+R₋/T₋^{½}]⁻²(z_{α}−z_{1-β})²σ_{R}²/σ_{A}².

The initial geometrical factor depends only on the pooling fraction. It is minimized at ρ=0.188 with a value of 2.90, yielding

N_{sib-radial}=2.90[R₊/T₊^{½}+R₋/T₋^{½}]⁻²(z_{α}−z_{1-β})²σ_{R}²/σ_{A}²

for this pooling design. The subsequent factor depends only on the phenotypic correlation t_{R} between siblings.

Sib-mean Design

The fraction ρ of the total population selected according to sib-mean pooling is defined in terms of the upper threshold X_{U} and the lower threshold X_{L} as

$$ρ = {\sum }_{{G}_{1} , {G}_{2}} P ( {G}_{1} , {G}_{2} ) Φ \{ - [ {X}_{U} - {μ}_{+} ( {G}_{1} , {G}_{2} ) ] / {σ}_{+} \} = {\sum }_{{G}_{1} , {G}_{2}} P ( {G}_{1} , {G}_{2} ) Φ \{ [ {X}_{L} - {μ}_{+} ( {G}_{1} , {G}_{2} ) ] / {σ}_{+} \} .$$

The genotype distribution describing the individuals selected for each pool follows a multinomial distribution based on sib-pair genotypes rather than individual genotypes,

$$1 = {\sum }_{{G}_{1} , {G}_{2}} {θ}_{U} ( {G}_{1} , {G}_{2} ) = {\sum }_{{G}_{1} , {G}_{2}} {θ}_{L} ( {G}_{1} , {G}_{2} ) , with \\ {θ}_{U} ( {G}_{1} , {G}_{2} ) {ρ}^{- 1} Φ \{ - [ {X}_{U} - μ ( G ) ] / {σ}_{R} \} P ( {G}_{1} , {G}_{2} ) and \\ {θ}_{L} ( {G}_{1} , {G}_{2} ) {ρ}^{- 1} Φ \{ [ {X}_{L} - μ ( G ) ] / {σ}_{R} \} P ( {G}_{1} , {G}_{2} ) .$$

The expected allele frequencies under the alternative hypothesis are

$$E ( {p}_{U} ) = {\sum }_{{G}_{1} , {G}_{2}} {θ}_{U} ( {G}_{1} , {G}_{2} ) {p}_{+} ( {G}_{1} , {G}_{2} ) and$$

$$E ( {p}_{L} ) = {\sum }_{{G}_{1} , {G}_{2}} {θ}_{L} ( {G}_{1} , {G}_{2} ) {p}_{+} ( {G}_{1} , {G}_{2} ) , with \\ E ( Δ p ) = E ( {p}_{U} ) - E ( {p}_{L} )$$

and p₊(G₁,G₂) the sib-mean allele frequency as defined previously. The terms giving the expected variance of the test statistic under the null and alternative hypothesis are

$${σ}_{0}^{2} = 2 s \{ {\sum }_{{G}_{1} , {G}_{2}} {P ( {G}_{1} , {G}_{2} ) [ {p}_{+} ( {G}_{1} , {G}_{2} ) ]}^{2} \} - 2 {sp}^{2} = 2 {sR}_{+} {σ}_{p}^{2} = 3 {σ}_{p}^{2} and \\ {σ}_{1}^{2} = s \{ {\sum }_{{G}_{1} , {G}_{2}} {[ {θ}_{U} ( {G}_{1} , {G}_{2} ) + {θ}_{L} ( {G}_{1} , {G}_{2} ) ] [ {p}_{+} ( {G}_{1} , {G}_{2} ) ]}^{2} \} - s ( {p}_{U}^{2} + {p}_{L}^{2} ) .$$

The factor s=2 accounts for the family structure, as n/s rather than n measurements of p₊ are averaged to determine the allele frequency of each pool. The variance under the null hypothesis may be derived directly from the sib-pair genotype frequencies, or more simply by noting that the variance of the mean allele frequency for a sib-pair is R₊σₚ², which is (¾) as large as the variance σₚ² for an individual. The variance for each pool is reduced by averaging over n/2 such terms, and multiplying by 2 for the number of pools yields 3σₚ². These equations may be solved numerically to obtain exactly the population size N required to detect association with specified power.

An analytical approximation follows the same derivation used for the unrelated-population design, except that individual genotypes are replaced by sib-pair genotypes, and individual phenotypes, phenotype offsets, and allele frequencies are replaced by their sib-mean analogs. The upper and lower pooling thresholds are

X_{U}=−X_{L}=−σ₊Φ⁻¹(ρ),

and the allele frequency difference between pools is

$$E ( Δ p ) = 2 {y}_{ρ} [ {\sum }_{{G}_{1} , {G}_{2}} P ( {G}_{1} , {G}_{2} ) {μ}_{+} ( {G}_{1} , {G}_{2} ) {p}_{+} ( {G}_{1} , {G}_{2} ) ] / {ρσ}_{+} = ( 2 {y}_{ρ} / ρ ) ( {R}_{+} / {T}_{+}^{1 / 2} ) {σ}_{p} {σ}_{A} / {σ}_{R} ,$$

where y_{ρ} is the standard normal density at deviate Φ⁻¹(ρ) as before. The contributions of the corresponding low-order terms in σ₁² cancel, and the variance of Δp is the same under both hypotheses. The population size required by the sib-mean design is

N_{sib-mean}=(sρ/2y_{ρ}²)(T₊/R₊)(z_{α}−z_{1-β})²σ_{R}²/σ_{A}².

As before, the factor ρ/y_{ρ}² is optimized with a pooling fraction of 0.27, yielding

N_{sib-mean}=2.47(T₊/R₊)(z_{α}−z_{1-β})²σ_{R}²/σ_{A}²

for the corresponding population size.

Sib-difference Design

Under the sib-difference design, a sib pair is selected if the sib-difference X₋ is larger in magnitude than a threshold X_{T},

$$2 ρ = {\sum }_{{G}_{1} , {G}_{2}} P ( {G}_{1} , {G}_{2} ) Φ \{ [ {μ}_{-} ( {G}_{1} , {G}_{2} ) - {X}_{T} ] / {σ}_{-} \} + {\sum }_{{G}_{1} , {G}_{2}} P ( {G}_{1} , {G}_{2} ) Φ \{ - [ {μ}_{-} ( {G}_{1} , {G}_{2} ) + {X}_{T} ] / {σ}_{-} \} .$$

In the first term, sibling 1 has the higher phenotype and is selected for the upper pool, and sibling 2 is selected for the lower pool. In the second term, the roles of the siblings are reversed. Multinomial distributions are defined as θ_{U}(G₁,G₂), the genotype probabilities for sib pairs in which sibling 1 enters the upper pool, and θ_{L}(G₁,G₂), when sibling 1 enters the lower pool, with normalization

$$1 = {\sum }_{{G}_{1} , {G}_{2}} \{ {θ}_{U} ( {G}_{1} , {G}_{2} ) + {θ}_{L} ( {G}_{1} , {G}_{2} ) \} .$$

This normalization implies that

θ_{U}(G₁,G₂)=(2ρ)⁻¹P(G₁,G₂)Φ{[μ₋(G₁,G₂)−X_{T}]/σ₋} and

θ_{L}(G₁,G₂)=(2ρ)⁻¹P(G₁,G₂)Φ{−[μ₋(G₁,G₂)+X_{T}]/σ₋}.

Due to symmetry, θ_{U}(G₁,G₂) and θ_{L}(G₂,G₁) are identical. The expected allele frequency difference between pools is

$$E ( Δ p ) = {\sum }_{{G}_{1} , {G}_{2}} 2 {θ}_{U} ( {G}_{1} , {G}_{2} ) p\_ ( {G}_{1} , {G}_{2} ) - {\sum }_{{G}_{1} , {G}_{2}} 2 {θ}_{L} ( {G}_{1} , {G}_{2} ) p\_ ( {G}_{1} , {G}_{2} ) ;$$

by symmetry, each term contributes E(Δp)/2. To calculate the variance of Δp, it is important to note that the normalization of θ_{U} and θ_{L} to ½ implies that the probabilities for a multinomial distribution are 2θ_{U} and 2θ_{L}, with both θ_{U} and θ_{L} equal to P(G₁,G₂)/2 under the null hypothesis. The terms giving the variance under the null hypothesis and the alternative hypothesis are

$${σ}_{0}^{2} = 2 s {\sum }_{{G}_{1} , {G}_{2}} P ( {G}_{1} , {G}_{2} ) {p\_}^{2} = 2 {sR\_σ}_{p}^{2} = {σ}_{p}^{2} and$$

$${σ}_{1}^{2} = 2 {\sum }_{{G}_{1} , {G}_{2}} [ 2 {θ}_{U} ( {G}_{1} , {G}_{2} ) + 2 {θ}_{L} ( {G}_{1} , {G}_{2} ) ] {p\_}^{2} - {E ( Δ p )}^{2} .$$

The value of θ₀² under the null hypothesis may be obtained more simply by noting that the allele frequency difference between two siblings has variance θₚ², and the measured allele frequency difference is the average of n such terms.

The population size required to detect association may be determined exactly by numeric calculation of the threshold value X_{T} as a function of the pooling fraction ρ. This value is then used to determine E(Δp), θ₀² and θ₁².

An analytic expression accurate when σ_{R}² is close to 1 may be derived using the same technique as for the previous pooling designs. The analytic estimate for the threshold value is

X_{T}=−σ₋Φ⁻¹(ρ)

and the allele frequency difference is

$$E ( Δ p ) = 2 {y}_{ρ} [ {\sum }_{{G}_{1} , {G}_{2}} P ( {G}_{1} , {G}_{2} ) μ\_ ( {G}_{1} , {G}_{2} ) p\_ ( {G}_{1} , {G}_{2} ) ] / ρ σ\_ = ( 2 {y}_{ρ} / ρ ) ( R\_ / {T\_}^{1 / 2} ) {σ}_{p} {σ}_{A} / {σ}_{R}$$

where yₚ is the standard normal density at deviate Φ⁻¹(ρ). The variance term θ₁² equals θ₀² to this order of approximation, and the population size required by the sib-difference design is

N_{sib-diff}=(sρ/2y_{ρ}²)(T₋/R₋)(z_{α}−z_{1-β})²σ_{R}²/σ_{A}².

The geometrical factor ρ/y_{ρ}²is minimized with a pooling fraction of 0.27, and

N_{sib-diff}=2.47(T₋/R₋)(z_{α}−z_{1-β})²σ_{R}²/σ_{A}²

is the corresponding population size.

Combined Sib-mean and Sib-difference Design

Because the sib-mean variables X₊ and p₊ are uncorrelated from the sib-difference variables X₋ and p₋, association tests based separately on these sets of variables are statistically independent and may be combined to achieve greater power even when the same unselected population is used and even when the same sib pairs are selected under both designs.

The combined test uses the measured value of Δp as an estimator for σ_{A}/σ_{R},

E(σ_{A}/σ_{R})=(T_{±}^{½}/R_{±})(ρ/2y_{ρ}σ_{ρ)}E(Δp),

with the + sign for the sib-mean test and the − sign for the sib-difference test. The variance of the estimator is the variance of Δp, obtained previously as 2sR_{±}σₚ²/n, multiplied by the square of the preceding terms, or

Var(σ_{A}/σ_{R})=(T_{±}/R_{±})sρ/2y_{ρ}²N.

This expression differs by the factor sR_{±} from a similar expression derived previously (Ollivier et al. 1997) that incorrectly neglects the contribution of family structure to the variance of Δp.

To form a combined estimator for σ_{A/σ}_{R}, the sib-mean and sib-difference estimators are summed with weights proportional to the inverse of the estimator variance. The variance of the combined estimator is

Var(σ_{A}/σ_{R})=(sρ/2y_{ρ}²N)[(R₊/T₊)+(R₋/T₋)]⁻¹.

The population size required using the combined estimator is

N_{comb}=(sρ/2y_{ρ}²)[(R₊/T₊)+(R₋/T₋)]⁻¹σ_{R}²/σ_{A}².

At the optimal pooling fraction of ρ=0.27, the prefactor (sρ/2y_{ρ}²) is 2.47. Since the variance of the individual estimators are identical under the null and alternative hypothesis, the population size for the combined estimator is simply the reciprocal of the sum of the reciprocal population sizes required for the individual estimators.

2.4 Regression Tests

Regression tests requiring individual genotyping provide a benchmark for the efficiency of tests on pooled DNA. A regression test assesses the significance of the model

Xᵢ=m(pᵢ−p)+εᵢ

where i labels an observation, Xᵢ is an observed phenotypic variable with mean 0, pᵢ is a observed genotypic variable with mean p, and εᵢ is the residual contribution not explained by the model. The phenotype and genotypic variables for a regression test are the individual Xᵢ and pᵢ values measured for N unrelated individuals and the sib-mean and sib-difference variable X_{±} and p_{±} for N/2 sib-pairs.

The expectation for m is 0 under the null hypothesis and is

E(m)=σ_{A}/σₚ

for either individuals or sib-pairs under the alternative hypothesis. The variance of the estimator, assumed identical under both hypotheses with negligible error when σ_{R}² is close to 1, is

Var(m)=(s/N)Var(εᵢ)/Var(pᵢ)=(s/N)(T/R)σ_{R}²/σₚ²,

where s=1 for unrelated individuals or 2 for sib-pairs, and T/R=1 for unrelated individuals and T_{±}/R_{±} for sib-mean and sib-difference variables. The corresponding population sizes required are

N_{regr}=s(T/R)(z_{α}−z_{1-β})²σ_{R}²/σ_{A}².

An estimator formed by combining the sib-mean and sib-difference estimators has a population size requirement of

N_{regr}=s[R₊/T₊+R₋/T₋]⁻¹(z_{α}−z_{1-β})²σ_{R}²/σ_{A}².

2.5 Computational Methods

Results for required population sizes were obtained numerically using computations converged to 1 part in 10⁶ (Press et al. 1997). Brent's root-finding algorithm was used to determine the threshold values X_{U} and X_{L} for the upper and lower pools for a given pooling design and pooling fraction ρ; Brent's optimization algorithm was then used to find the ρ with maximum power. While the reported results are based on a normal approximation for the allele frequency difference Δp, results were also obtained for the exact multinomial distribution for the unrelated-population design. The difference between the numerical results for the multinomial and normal distributions was typically less than 1%. The population size required for the pooling combined estimator was obtained numerically as the reciprocal of the sum of the reciprocal exact sizes required for the sib-mean and sib-difference pooling designs. Using a 750 MHz Pentium III running Linux, the root-finding and minimization for each parameter set required less than 0.01 sec each design.

3. Results

3.1 Comparisons with Individual Genotyping

When the effect of a QTL is small and the residual variance σ_{R}² is close to 1, the analytic expressions for population size requirements are exact. In this limit, we begin by comparing the efficiency of pooled DNA designs relative to individual genotyping.

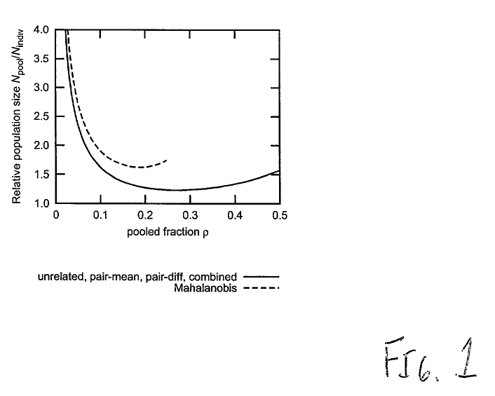

The population size requirements of pooled DNA methods are shown in FIG. 1 relative to corresponding regression tests for the same family structure for the unrelated-population, sib-mean, sib-difference, and combined designs, as well as the sib-radial design which will be discussed separately below. The ratio of population size requirements is independent of all model parameters except for the fraction p of individuals whose DNA is pooled. Furthermore, the ratio is independent of family structure for these matched comparisons. The pooling fraction ρ=0.27 is seen to be optimal. The curve is flat near the minimum, indicating that pooling fractions close to 0.27 give near-optimal results. For these designs, population sizes must be increased by 1.24× to attain the same power as would have been achieved with N individual genotypes.

The population size required for the sib-radial design is shown relative to that required for the most powerful regression test of sib pairs, the combined sib-mean and sib-difference estimator. This ratio depends on the residual phenotypic correlation t_{R} between siblings, and a typical value t_{R}=0.5 has been selected for illustrative purposes. The minimum occurs at 0.188 independent of t_{R}, with a population size 1.55× larger than that required for individual genotyping. Stated differently, when N genotypes of N/2 sib-pairs are replaced by 4 measurements of sib-mean and sib-difference pools, the population size requirement increases by about 25%; when the 4 pools are replaced by 2 pools with extreme individuals, the population size requirement increases by another 25%.

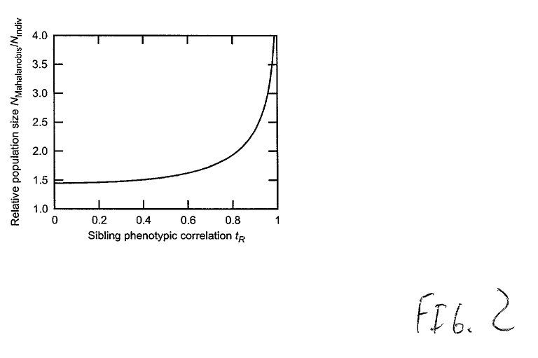

In FIG. 2, the performance of the sib-radial design relative to the combined regression test for individual genotypes is shown as a function of the residual sibling phenotypic correlation t_{R}, with the optimal fraction 0.188 used to construct the upper and lower pools. The ratio of population sizes is roughly 1.5 until the phenotypic correlation rises above 0.6, at which point the population size requirements for the sib-radial design begin to rise more steeply.

3.2 Comparisons Between Sibling and Unrelated Populations

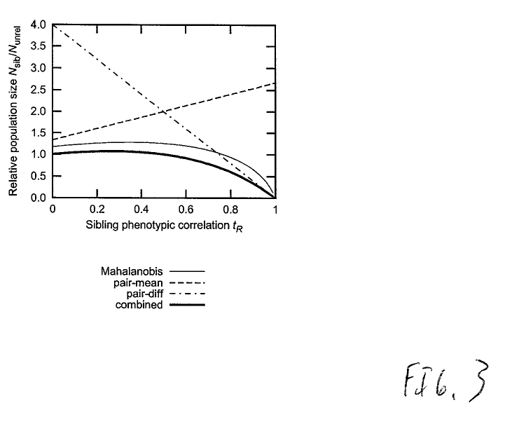

In FIG. 3, the population size requirements for association tests using DNA pooled from sib pairs are shown as a function of the residual sibling phenotypic correlation t_{R}, relative to the population size required for a test of DNA pooled from unrelated individuals. Ratios larger than 1 indicate that the population of N unrelated individuals is more powerful than a population of N/2 sib pairs, while ratios smaller than 1 indicate that the sib-pair population is more powerful. These ratios are derived from the appropriate analytic expressions in the limit of a QTL making a small contribution to a complex trait.

The combined test using sib-mean and sib-difference pools is uniformly the most powerful sib-pair design for all values of t_{R}. Its worst-case performance relative to an unrelated population occurs when t_{R} is (3^{½}+1)/(3^{½}−1), or 0.2679, where it requires a population 7% larger. The unrelated and sib-pair tests require the same population size when the phenotypic correlation is 0.5, and the sib-pair test becomes much more powerful for equal population sizes for larger values of t_{R}.

The sib-pair designs requiring only a single pair of pools, a population of unrelated individuals is more powerful than a population of sib pairs except for large values of the sibling phenotypic correlation, t_{R}>0.75, at which point the sib-radial and sib-difference designs become more powerful. Below this phenotypic correlation, the sib-radial design is substantially more powerful than the other sib-pair tests; above this correlation, the sib-difference design is only slightly more powerful than the sib-radial design. As t_{R} increases, the sib-mean design decreases in power and the sib-difference design increases in power.

3.3 Sensitivity to QTL Contribution, Allele Frequency, Inheritance Mode

According to the analytic theory, the population size requirements for pooling tests is inversely proportional to the additive variance contributed by the QTL relative to the residual phenotypic variance, σ_{A}²/σ_{R}², and independent of any remaining parameters of the genetic model. Here we provide exact numerical results to assess the region of validity for the analytic results.

For these numerical results, the type I error rate α is 5×10⁻⁸ and the type II error rate β is 0.2; these values have been suggested for whole-genome scans (Risch and Merikangas 1996). The sibling phenotypic correlation t_{R} was also held fixed for the numerical tests. Estimates of the genetic heritability for complex traits range from 20% for cancer (Verkasalo et al. 1999), 20% to 40% for Type 2 diabetes mellitus (NIDDM) (Watanabe et al. 1999), 50% for pulmonary function (Wilk et al. 2000), 10% to 50% for systolic and diastolic blood pressure (Iselius et al. 1983, Perusse 1989), and 70% to 90% for cholesterol level (Austin et al. 1987). Shared environmental factors are estimated to contribute 7% of the overall phenotype variance for cancer (Verkasalo et al. 1999), 20% to 40% for blood pressure (Iselius et al. 1983, Perusse et al. 1989), and 15% for serum lipid levels (Heller et al. 1993). The sibling phenotype correlation, equal to half the genetic heritability plus the shared environmental contribution, varies over a wide range for these traits. An intermediate value of t_{R}=0.6 was selected. Choosing a different value of t_{R} changes the relative power of different pooling designs, as shown in FIG. 3, but does not alter any conclusions regarding the validity of the analytic theory.

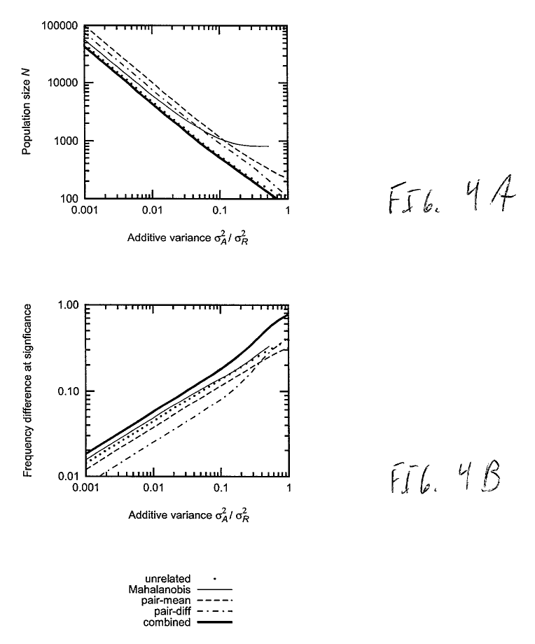

In FIG. 4A, the population size required by each pooling design is shown as a function of σ_{A}²/σ_{R}² the QTL additive variance relative to the residual phenotypic variance 1−σ_{A}²−σ_{D}². The QTL has purely additive inheritance and the minor allele frequency is 0. 1. The minor allele frequency of 0.1I used in this example is typical for polymorphisms in coding regions. Reported minor-allele frequencies for SNPs found in multiple populations range from 0.05 to 0.25, with lower frequencies for variations which cause non-conservative amino acid changes and higher frequencies for conservative substitutions and changes in non-coding regions (Cargill et al. 1999, Goddard et al. 2000).

In this and all subsequent figures, the unrelated-population design is a dotted line, sib-radial is a thin line, sib-mean is dashed, sib-difference is dot-dashed, and the combined estimator sib-comb is a thick line. For FIG. 4, the pooling fractions have been optimized for maximum power. Linearity in the log-log plot demonstrates validity of the analytic results, with optimized pooling fractions close to 0.27 or 0. 1 88 for the sib-radial design and population sizes accurately predicted. Inspection of the results shows that agreement extends almost to σ_{A}²/σ_{R}²=1, where the QTL is responsible for half the phenotypic variance, for all the designs except sib-radial. The sib-radial design is less powerful than predicted by analytic theory for σ_{A}²/σ_{R}²>0.05, roughly the transition between a complex trait and a monogenic trait.

The allele frequency difference at the significance threshold, z_{α}σ₀/n^{½}, is shown in FIG. 4B for the same set of parameters. As the QTL contribution is smaller, allele frequency differences must be measured with precision. While raw frequency differences of 10% are significant for a monogenic trait, σ_{A}²/σ_{R}²˜0.1, raw frequencies differences of 3% must be measured with little error to achieve maximum power for a complex trait with σ_{A}²/σ_{R}²˜0.01.

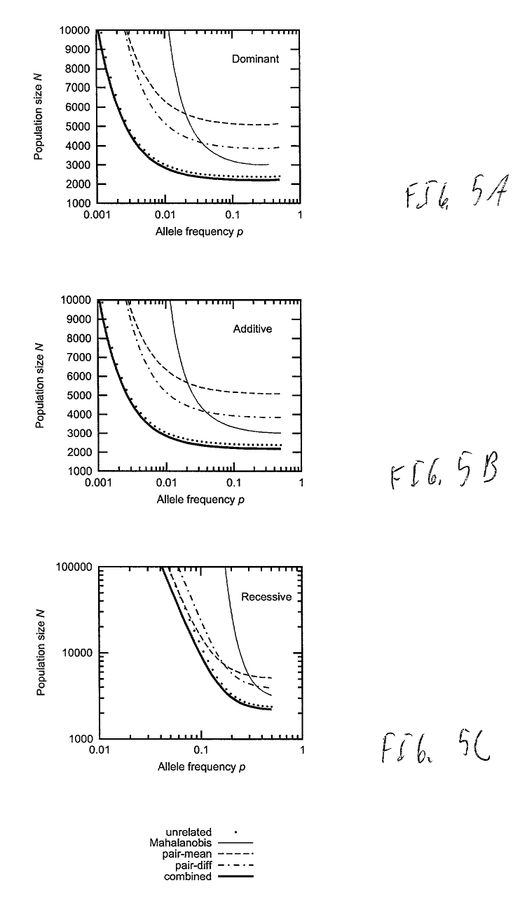

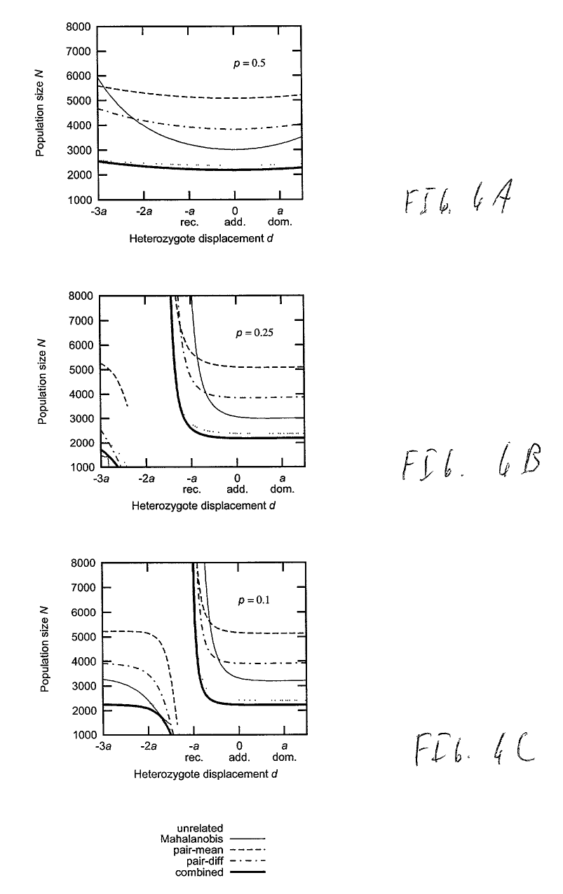

The sensitivity of the results to both the allele frequency p and the inheritance mode d/a is shown in FIGS. 5 and 6. In both of these figures, the pooling fractions are fixed at the limiting values 0.27 for the unrelated-population, sib-mean, sib-difference, and sib-combined designs and at 0.188 for the sib-radial design, as would be presumably be done if DNA is pooled once then used repeatedly in a genome-wide screen of markers. In FIG. 5, the allele frequency is varied for a phenotype with a dominant inheritance (FIG. 5A), additive inheritance (FIG. 5B), and recessive inheritance (FIG. 5C) with respect to the minor allele. The QTL contribution σ_{A}²/σ_{R}² is held fixed at 0.02 for these comparisons. The figures are shown only for the region p<0.5 to highlight the behavior for small values of p; additive alleles are symmetric about p=0.5, while dominant major alleles are equivalent to recessive minor alleles and vice versa.

The population size is rather insensitive to allele frequency for p>0.01 for dominant and additive inheritance, and for p>0.2 for recessive inheritance, for all but the sib-radial design, indicating that the analytic theory is valid in these regions. The population size required to detect association increases rapidly as the allele frequency decreases below these limits. The sib-radial design is more sensitive to the allele frequency than the other designs, losing power rapidly as the allele frequency falls below 0.1 for dominant and additive inheritance and 0.2 for recessive inheritance.

The allele frequency at which the analytic theory loses accuracy may be estimated by noting that the perturbation parameters used to derive the theory are the terms μ(G)/σ_{R}. When these terms grow close to 1 or larger, the perturbation theory becomes less reliable. For a complex trait, σ_{R} is close to 1, and the theory is unreliable when a or d is close in magnitude to 1. This occurs for p=σ_{A}²/8 under dominant inheritance, σ_{A}²/2 under additive inheritance, and σ_{A}^{⅔}/2 for recessive inheritance. In FIG. 5, these values are 0.0025, 0.01, and 0.14, and accurately identify the elbows of the population size curves.

In FIG. 6, the inheritance mode is varied while the allele frequency is held fixed at one of three values, p=0.5 (FIG. 6A), 0.25 (FIG. 6B), or 0.1 (FIG. 6C). When p=0.5, the inheritance mode has virtually no effect on the population size required to detect association. The sib-radial design is an exception, with increasing requirements only for strong over-dominance. For p<0.5, the additive variance necessarily vanishes at d/a=1/(2p−1); when d/a is close to this value, the population requirements increase dramatically. For p=0.25, this occurs at d/a=−2. Above this critical value of d/a, excess of A₁A₁ homozygotes are detected in the upper pool; below the critical value, excess A₁A₂ heterozygotes are detected in the lower pool. Although Δp is negative in this region and therefore not significant under a one-sided test of allele A₁, the test of A₂ would be significant. Curiously, the population size requirements are substantially smaller than predicted by analytic theory for this region of strongly over-dominant major alleles.

In the bottom panel, FIG. 6C, the allele frequency is p=0.1 and the critical value of d/a is −1.125. The region of increased population requirements is narrower than in FIG. 6B, and becomes narrower still when p is further reduced, but the general behavior is the same.

We have also investigated the sensitivity of the exact numerical results to specified rates of type I and type II error. The behavior is predicted nearly exactly by the term (z_{α}−z_{1-β})² (results not shown). Using a fixed power of 0.8(z_{1-β}=−0.84), for example, a whole-genome scan with α=5×10⁻⁸ (z_{α}=5.33) requires 1.7× more individuals than a test of 1000 candidate markers with α=5×10⁻⁵ (z_{α}=3.89) and 6.2× more individuals than a test of a single marker with α=0.05 (z_{α}=1.64).

References

Abecasis, G R, Cardon, L R, Cookson, W O C (2000) A general test of association for quantitative traits in nuclear families. Am J Hum Genet 66: 279-292.

Austin M A, King M C, Bawol R D, Hulley S B, Friedman G D (1987) Risk factors for coronary heart disease in adult female twins. Genetic heritability and shared environmental influences. Am J Epidemiol 125: 308-18.

Barcellos L F, Klitz W, Field L L, Tobias R, Bowcock A M, Wilson R, Nelson M P et al. (1997) Association mapping of disease loci, by use of a pooled DNA genomic screen. Am J Hum Genet 61:734-747.

Beyer W H (ed) (1984) CRC Standard Mathematical Tables, 27^{th} Edition. CRC Press, Boca Raton, Fla.

Cargill M, Altshuler D, Ireland J, Sklar P, Ardlie K, Patil N, Shaw N et al. (1999) Characterization of single-nucleotide polymorphisms in coding regions of human genes. Nat Genet 1999 July; 22(3):231-238.

Cardon L R (2000) A Sib-Pair Regression Model of Linkage Disequilibrium for Quantitative Traits. Hum Hered. 50:350-358.

Collins A, Lonjou C, Morton N E (2000) Genetic epidemiology of single-nucleotide polymorphisms. Proc Natl Acad Sci USA 96:15173-15177.

Darvasi A, Soller M (1994) Selective DNA pooling for determination of linkage between a molecular marker and a quantitative trait locus. Genetics 138: 1365-1373.

Falconer, D S, MacKay, T F C (1996) Introduction to quantitative genetics. Addison-Wesley, Boston.

Fulker DW, Cherny S S, Cardon L R (1995) Multipoint interval mapping of quantitative trait loci, using sib pairs. Am J Hum Genet 56:1224-1233.

Frank, L (2000) Storm brews over gene bank of Estonian population. Science 286: 1262.

Goddard K A, Hopkins P J, Hall J M, Witte J S (2000) Linkage disequilibrium and allele-frequency distributions for 114 single-nucleotide polymorphisms in five populations. Am J Hum Genet 66:216-34.

Gu C, Todorov A, Rao D C (1996) Combining extremely concordant sibpairs with extremely discordant sibpairs provides a cost effective way to linkage analysis of quantitative trait loci. Genet Epidemiol 13:513-533.

Heller D A, de Faire U, Pedersen N L, Dahlen G, McClearn G E (1993) Genetic and environmental influences on serum lipid levels in twins. N Engl J Med 328: 1150-6.

Iselius L, Morton N E, Rao D C (1983) Family resemblance for blood pressure. Hum Hered 33: 277-286.

Kruglyak, L (1999) Prospects for whole-genome linkage disequilibrium mapping of common disease genes. Nature Genetics 22: 139-144.

Kruglyak L, Lander E S (1995) Complete multipoint sib-pair analysis of qualitative and quantitative traits. Am J Hum Genet 57:439-454.

Liu, B-H (1997) Statistical Genomics. CRC Press, Boca Raton.

Mathews J, Walker R L (1970) Mathematical methods of physics, second edition. Benjamin/Cummings, London.

Neale, M C and Cardon, L R (1992). Methodology for Genetic Studies of Twins and Families, NATO ASI Series D, Behavioural and Social Sciences, Vol. 67. Kluwer Academic, Dordrecht.

Nilsson A, Rose J (1999) Sweden takes steps to protect tissue banks. Science 286: 894.

Ott J (1999) Analysis of human genetic linkage. Johns Hopkins Univ Pr, Baltimore.

Perusse L, Rice T, Bouchard C, Vogler G P, Rao D C (1989) Cardiovascular risk factors in a French-Canadian population: resolution of genetic and familial environmental effects on blood pressure by using extensive information on environmental correlates. Am J Hum Genet 45: 240-251.

Press, W H, Teukolsky, S A, Vetterling, W T, and Flannery, B P (1997) Numerical Recipes in C, The Art of Scientific Computing, Second Edition. Cambridge University Press, Cambridge, UK.

Rabinow, P (1999) French DNA: Trouble in Purgatory. University of Chicago Press, Chicago.

Risch N J (2000) Searching for genetic determinants in the new millennium. Nature 405: 847-856.

Risch N J, Merikangas K (1996) The future of genetic studies of complex human diseases. Science 273:1516-1517.

Risch N J, Teng J (1998) The relative power of family-based and case-control designs for linkage equilibrium studies of complex human diseases I. DNA pooling. Genome Res 8:1273-1288.

Risch N J, Zhang H (1996) Mapping quantitative trait loci with extreme discordant sib pairs: sampling considerations. Am J Hum Genet 58:836-843.

Sham, P (1997) Statistics in Human Genetics. Arnold.

Verkasalo P K, Kaprio J, Koskenvuo M, Pukkala E (1999) Genetic predisposition, environment and cancer incidence: a nationwide twin study in Finland, 1976-1995. Int J Cancer 83: 743-749.

Watanabe R M, Valle T, Hauser E R, Ghosh S, Eriksson J, Kohtamaki K, Ehnholm C et al. (1999) Familiality of quantitative metabolic traits in Finnish families with non-insulin-dependent diabetes mellitus. Finland-United States Investigation of NIDDM Genetics (FUSION) Study investigators. Hum Hered 49: 159-168.

Wilk J B, Djousse L, Arnett D K, Rich S S, Province M A, Hunt S C, Crapo R O et al. (2000) Evidence for major genes influencing pulmonary function in the NHLBI family heart study. Genet Epidemiol 19: 81-94.

Zhang H, Risch N (1995) Extreme discordant sib pairs for mapping quantitative trait loci in humans. Science 268:1584-1589.

Zhang H, Risch N (1996) Mapping quantitative-trait loci in humans by use of extreme concordant sib pairs: selected sampling by parental phenotypes. Am J Hum Genet 59:951-957.

Tables

TABLE I
Sib-pair genotype probabilities

Sib Genotype

| G₁ | G₂ | P(G₁, G₂) |
|---|---|---|
| A₁A₁ | A₁A₁ | p⁴ + p³(1 − p) + p²(1 − p)²/4 |
| A₁A₁ | A₁A₂ | p³(1 − p) + p²(1 − p)²/2 |
| A₁A₁ | A₂A₂ | p²(1 − p)²/4 |
| A₁A₂ | A₁A₁ | p³(1 − p) + p²(1 − p)²/2 |
| A₁A₂ | A₁A₂ | p³(1 − p) + 3p²(1 − p)² + p(1 − p)³ |
| A₁A₂ | A₂A₂ | p²(1 − p)²/2 + p(1 − p)³ |
| A₂A₂ | A₁A₁ | p²(1 − p)²/4 |
| A₂A₂ | A₁A₂ | p²(1 − p)²/2 + p(1 − p)³ |
| A₂A₂ | A₂A₂ | p²(1 − p)²/4 + p(1 − p)³ + (1 − p)⁴ |

Other Embodiments

Additional embodiments are within the claims.

What is claimed is:

1. A method for detecting an association in a population of individuals between a genetic locus and a quantitative phenotype, wherein two or more alleles occur at the locus, and wherein the phenotype is expressed using a numerical phenotypic value whose range falls within a first numerical limit and a second numerical limit, the method comprising the steps of
a) obtaining the phenotypic value for each individual in the population; wherein said population comprises sibling pairs;
b) selecting a first subpopulation of individuals having phenotypic values that are higher than a predetermined lower limit and pooling DNA from the individuals in the first subpopulation to provide an upper pool;
c) selecting a second subpopulation of individuals having phenotypic values that are lower than a predetermined upper limit and pooling DNA from the individuals in the second subpopulation to provide a lower pool;
d) for one or more genetic loci, measuring the difference in frequency of occurrence of a specified allele between the upper pool and the lower pool; and
e) determining that an association exists if the allele frequency difference between the pools is larger than a predetermined value.

2. The method described in claim 1 wherein the phenotypic value is obtained as a numerical combination of other phenotypic values.

3. The method described in claim 2 wherein the phenotypic values are obtained from regressing out the effect of age.

4. The method described in claim 1 wherein the phenotypes are numerical rankings.

5. The method described in claim 1 wherein the lower limit and the upper limit are chosen such that, for a specified false-positive rate, the frequency of occurrence of false-negative errors is minimized.

6. The method described in claim 1 wherein the population comprises unrelated individuals.

7. The method described in claim 6 wherein the predetermined lower limit is set so that the upper pool includes the highest 35% of the population and the predetermined upper limit is set so that the lower pool includes the lowest 35% of the population.

8. The method described in claim 6 wherein the predetermined lower limit is set so that the upper pool includes the highest 30% of the population and the predetermined upper limit is set so that the lower pool includes the lowest 30% of the population.

9. The method described in claim 6 wherein the predetermined lower limit is set so that the upper pool includes the highest 27% of the population and the predetermined upper limit is set so that the lower pool includes the lowest 27% of the population.

10. The method described in claim 1 wherein each family is considered as a unit, and either (i) both sibs are selected for the upper pool; (ii) both sibs are selected for the lower pool; or (iii) neither sib is selected.

11. The method described in claim 10 wherein selection is based on the mean phenotype of the two sibs.

12. The method described in claim 11 wherein selection is based on both sibs being above a threshold or below a threshold.

13. The method described in claim 6 wherein the individuals in the population are sibling pairs and each pair is ranked according to a mean value of the phenotypic values of the siblings in each pair, and for sibling pairs that are in a pool, both members of the sibling pair are in the same pool.

14. The method described in claim 10 wherein the predetermined lower limit is set so that the upper pool includes the pairs with highest 35% of the mean values in the population and the predetermined upper limit is set so that the lower pool includes the lowest 35% of the mean values in the population.

15. The method described in claim 10 wherein the predetermined lower limit is set so that the upper pool includes the highest 30% of the mean values in the population and the predetermined upper limit is set so that the lower pool includes the lowest 30% of the mean values in the population.

16. The method described in claim 10 wherein the predetermined lower limit is set so that the upper pool includes the highest 27% of the mean values in the population and the predetermined upper limit is set so that the lower pool includes the lowest 27% of the mean values in the population.

17. The method described in claim 1 wherein each sib-pair is considered as a unit, and either (i) one sib is selected for the upper pool, and the other sib is selected for the lower pool; or (ii) neither sib is selected.

18. The method described in claim 17 wherein selection is based on the magnitude difference between sib phenotype values.

19. The method described in claim 17 wherein selection is based on one sib being above a threshold and the other sib being below a threshold.

20. The method described in claim 6 wherein the individuals in the population are sibling pairs, the pairs are ranked by the absolute magnitude of the difference in phenotypic value for the sibs within each pair, the percent of pairs with greatest difference are identified, the percent of pairs being 70%, and the siblings in each pair are distributed such that the sibling with the high phenotypic value is selected for the upper pool and the sibling with the low phenotypic value is selected for the lower pool, providing 35% of the population in each pool.

21. The method described in claim 20 wherein the percent of pairs is 60% and the distribution provides 30% of the population in each pool.

22. The method described in claim 20 wherein the percent of pairs is 54% and the distribution provides 27% of the population in each pool.

23. The method described in claim 6 wherein the individuals in the population are sibling pairs.

24. The method described in claim 1 wherein an unrelated population is selected from a sib-pair population and pooling is conducted on the derived unrelated population.

25. The method described in claim 24 wherein the sibling with phenotype furthest from the overall mean is selected from each family to generate an unrelated population.

26. The method described in claim 6 wherein the unrelated individuals are provided by a process comprising the steps of:
a) providing a superpopulation of individuals, each individual being a member of a sibling pair;
b) selecting that member of each sibling pair having a phenotypic value such that the absolute value of the difference between the individual's phenotypic value and either the first numerical limit or the second numerical limit is lower than the difference for the other individual in the pair, thus providing a population of unrelated individuals;
c) setting the predetermined lower limit so that the upper pool includes the highest 36% of the population and the setting the predetermined upper limit so that the lower pool includes the lowest 36% of the population.

27. The method described in claim 1 wherein
(i) one member of each sibling pair is chosen at random to provide a group of unrelated individuals; and
(ii) the members of the group having phenotypic values greater than a predetermined lower limit are placed in the first subpopulation and the members of the group having phenotypic values lower than a predetermined upper limit are placed in the second subpopulation.

28. The method described in claim 1 wherein only one member of a sibling pair is placed in a subpopulation; wherein the fraction of individuals in the first subpopulation is determined using Equation A and the fraction of individuals in the second subpopulation is determined using Equation B, and wherein the sibling with genotype G₁ is selected for the upper pool if the value of φ is in the interval 0<φ<π/2 or is selected for the lower pool if the value of φ is in the interval π<φ<3π/2 and the sibling with genotype G₂ is selected otherwise.

29. The method described in claim 1 wherein
(i) the mean phenotypic value for the pair is calculated; and
(ii) the first subpopulation contains those pairs whose mean phenotypic value is greater than a predetermined minimum value and the second subpopulation contains those pairs whose mean phenotypic value is lower than a predetermined maximum value.

30. The method described in claim 1 wherein
(i) the difference between the phenotypic values for the members of each sibling pair is calculated;
(ii) those sibling pairs whose values of the calculated difference are greater than a predetermined minimum value for the difference are identified; and
(iii) in each identified sibling pair, placing the sibling with the higher phenotypic value in the first subpopulation and the sibling with the lower phenotypic value in the second subpopulation.

31. The method described in claim 1 wherein:
(i) the mean phenotypic value for the pair is calculated; and
(ii) a first upper subpopulation contains those pairs whose mean phenotypic value is greater than a predetermined minimum value and a first lower subpopulation contains those pairs whose mean phenotypic value is lower than a predetermined maximum value;
(iii) the difference between the phenotypic values for the members of each sibling pair is calculated;
(iv) those sibling pairs whose values of the calculated difference of step (iii) are greater than a predetermined minimum value for the difference are identified; and
(v) in each sibling pair identified in step (iv), placing the sibling with the higher phenotypic value in a second upper subpopulation and the sibling with the lower phenotypic value in a second lower subpopulation.
